(19) United States
(12) Patent Application Publication
(10) Pub. No.: US 2024/0423826 A1
(43) Pub. Date: Dec. 26, 2024

(54) Jaw Bridge

(71) Applicant: Andrew Kohler, Amissville, VA (US)

(72) Inventor: Andrew Kohler, Amissville, VA (US)

(21) Appl. No.: 18/341,117

(22) Filed: Jun. 26, 2023

(51) Int. Cl. A61F 5/56; A61F 5/01

(52) U.S. Cl. CPC A61F 5/56; A61F 5/01

(57) ABSTRACT

This device is a mechanical therapeutic intervention to support the customer's head at rest, thereby preventing airway obstruction. This treats the disease process of OSA, a major cause of morbidity and mortality in our world. The technical components of this device include the importance of structural stability in maintaining the position once in place, mobility of the head, no impingement and compromise to the vital structures of the anterior neck, as well as structural strength when the weight of the head is applied. The concept, and overall design structure to avoid at-risk anatomic structures and promote wearability, is unique in design.

### CROSS-REFERENCE TO RELATED APPLICATIONS

US-20210275226-A1 Kaveh; Cameron 09-2021
U.S. Ser. No. 10/117,768-B1 Freed; William L. 11-2018
US-20140296653-A1 Jenkins, III; Arthur J. 10-2014
U.S. Pat. No. 7,371,221-B1 Baker, Ford S. 05-2008
U.S. Pat. No. 5,385,535-A McGuinness; Charles 01-1995
U.S. Pat. No. 3,306,284-A McKinley; Paul E. 02-1967

It is not obvious that these prior art applications/patents accomplish the same goal. The prior art appears to stabilize the cervical spine, while JawBridge facilitates mobility and remains clear of vital anterior neck structures.

### STATEMENT REGARDING FEDERALLY SPONSORED RESEARCH OR DEVELOPMENT

This application is not part of Federally sponsored research or development.

### THE NAMES OF THE PARTIES TO A JOINT RESEARCH AGREEMENT

Andrew F Kohler only

### INCORPORATED-BY-REFERENCE MATERIAL

Submitted as figures through the Patent Office Electronic System.

### PRIOR DISCLOSURES

None

### BACKGROUND ON THE PROBLEM AND THE JAWBRIDGE SOLUTION

Obstructive Sleep Apnea (OSA) impairs the individual's ability to breathe at night. This prevents an adequate night's rest and results in increased CO2 levels in the body, contributing to chronic conditions of fatigue, cardiovascular disease, diabetes, obesity, cerebrovascular accidents, and early death.

Multiple factors play a role, but soft tissue of the neck collapsing on the airway during sleep is a primary causation. This also leads to the phenomenon of snoring. The device treats mild and moderate cases of obstructive sleep apnea with secondary benefits as a remedy to snoring and utilization during procedural sedation. The invention stems from this mechanical condition lacking an adequate mechanical correction by current technology.

This device is the mechanical fix for a mechanical problem. It is not obvious that other devices address the problem at the source, jaw and soft tissue collapse. The field's current gold standard is the cumbersome CPAP machine, with documented poor compliance. This previous method of OSA intervention is a modality of positive pressure with an external, invasive device. The CPAP device will now be reserved for the more extreme case, for example in instances of larger body habitus, while JawBridge will provide relief for mild to moderate cases.

This device prevents OSA, thereby treating snoring and mitigating the risk of downstream sequalae: fatigue, weight gain, hypertension, CVD, strokes, heart attacks, early death. The angulation and design are based on medical specialist feedback and engineering with expert training in the medical field to ensure no nerve, vascular, or muscle bed body is impinged upon.

### Individual and Business Demographics that would Use this Invention

Private citizens with mils and moderate OSA, those afflicted by snoring, procedural sedation in healthcare institutions or outpatient medical facilities.

### BRIEF SUMMARY OF THE INVENTION

The device design is a cradle for the jaw, maintaining the neutral position held while awake, thereby preventing the soft tissue collapse that happens during sleep. Taking the shape of an “X” draped behind the neck, with the center at the most prominent spinous process between the clavicles (shoulder blades) (C7). The tops of the X reach upward and forward, meeting the mandible cradle process to support the angle and along the body of the mandible (a cushioned surface will contact the skin). The two “feet” of the X extend downward and angle forward, approximating the shoulders and clavicles as the foundation strut.

The device is anatomically designed to avoid the neck's vital structures around the jaw and no component contacts the front of the neck. A gear in the center manually raises the top arms into position once placed on the nape of the neck. The device is wrapped in a removable cushion for comfort and washing purposes.

Correct placement is one of a neutral head alignment, no hyperextended “sniffing position” is required. It is also anticipated that this device will replace the need for an anesthesiologist's protracted manual hold and OR/ER procedural sedation airway positioning. Manufacturing entails two sizes for large and small size options.

### DESCRIPTION OF INVENTION DRAWINGS

Illustration Brief Description:

FIG. 1: Overall design render

FIG. 2: Positioning on customer

The correct placement is one of a neutral head alignment, with clavicle processes resting on the clavicles and mandible process approximating the angle of the jaw

FIG. 3: Positioning on customer

FIG. 4: Positioning on customer

FIG. 5: Positioning on customer

FIG. 6: Final prototype utilized in pilot program

FIG. 7: Final prototype utilized in pilot program

FIG. 8: Final prototype utilized in pilot program

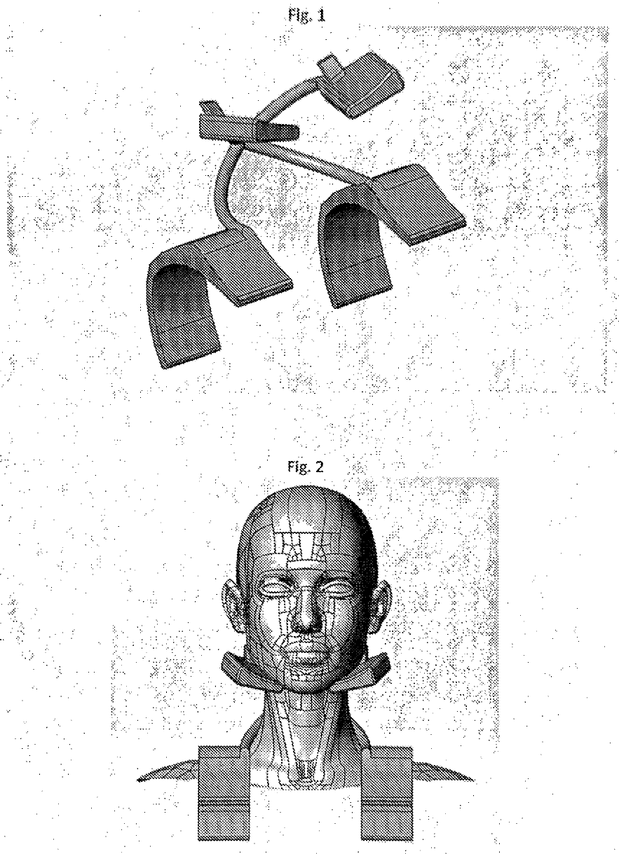

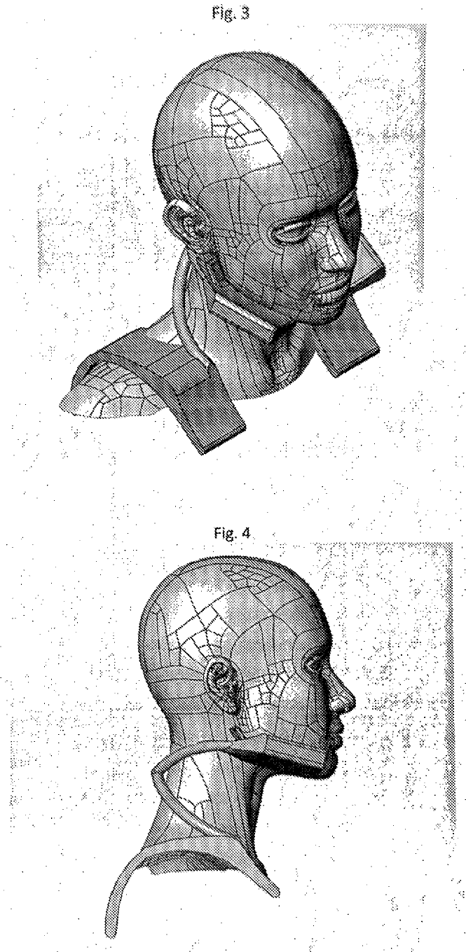

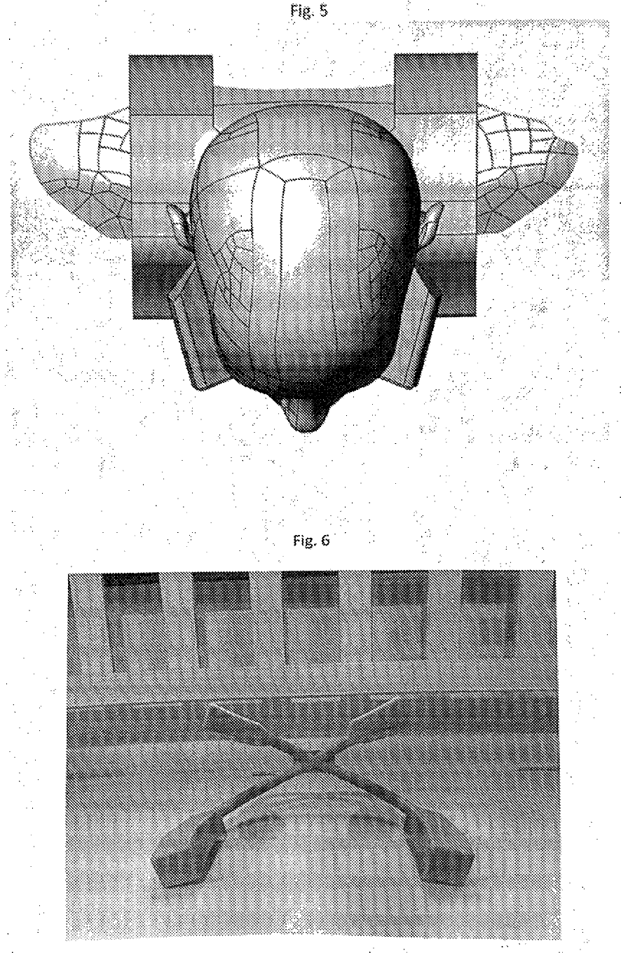

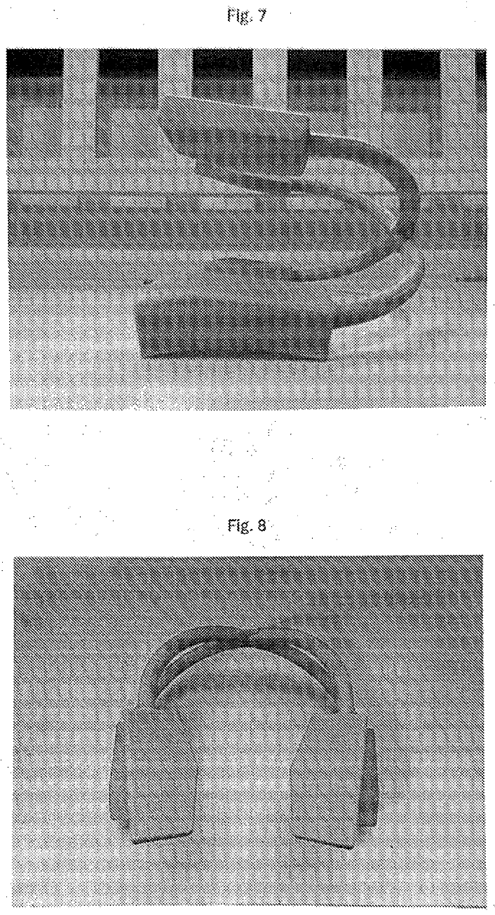

### PARTS AND COMPONENTS IDENTIFICATION

Each part and component of the Invention with assigned reference number and title

| REFERENCE | |
|---|---|
| NUMERAL | PART NAME |
| 1 | Structural body X |
| 2 | Clavicle process |
| 3 | Gear |
| 4 | Mandible process |
| 5 | Pillow (Cushion and cover) |

### INVENTION DETAILED DESCRIPTION

### Detailed Description of the Parts of the Invention

| PART | |
|---|---|
| NUMBER | PART DESCRIPTION |
| 1 | The structure will take the shape of an “X” positioned behind the |
| | neck, with the center extending through the gear box. This cross- |
| | section will anatomically occur immediately above the most |
| | prominent spinous process between your shoulder blades (C7). |
| | The two tops of the X will reach upward and forward to cradle the |
| | jaw (a cushioned surface will protect the skin). The two lower arms |
| | of the X structure will extend downward and at a curved angle |
| | forward, approximating the clavicles. These lower arms function as |
| | the support struts. |
| 2 | The two “feet” processes of the X will approximate the shoulders/ |
| | clavicles (shoulder blades) to stabilize the X structural body. The |
| | clavicle process itself will extend outward from center (2inches). |
| | The body of the process will take a semi-circular, cylindrical form, |
| | 120 degrees in circumference. The process will approximate to |
| | the clavicle along the anterior (front) and superior (top) aspects, |
| | extending over the trapezius muscle. The entire shoulder. |
| 3 | The gear will be circular in form, 1 inch in diameter. The action of manually |
| | turning the gear will raise the two beams which create the “X” structure. |
| | The arms of the X will extend in one full length through the gear for |
| | maximum strength, with the action of the gear upon the central portion of |
| | the arms, moving them counter to each other, thereby shortening or |
| | lengthening the X as desired by the user. Tightening the gear will result in a |
| | change from a flattened, elongated (acute lateral angle) X to a taller (more |
| | obtuse lateral angle) X. This gear will be within the cloth cushion and lock in |
| | the position as adjusted by the user for comfort and effectiveness. |
| 4 | The mandible process formation cradles the angle of the jaw. Taking the |
| | shape of an “L” laying on its back, the long arm (3 inches) will rest along the |
| | jaw line. The short arm (2 inches) will reach along the back of the jaw, |
| | extending to the preauricular area at the front aspect of the ear. The “L” will |
| | be at an 80 degree angle from the head's apex being 0 degrees, to each arm |
| | and to the “X” structure. The “L”s will be 1 inch in width. The inner aspect |
| | of the L (that which is approximating to the skin surface) will have a layer of |
| | ½ inch foam polymer affixed to the superior surface. |
| 5 | The cloth cushion is of washable cotton and padding materials, form fitted |
| | to the device, to provide a protective coating for the purposes of comfort. |

### Relationship Between the Parts of the Invention

The clavicle process will be one continuous unit affixed to the bases of the X structure. The mandible process represents the upper arm endings of the X structure, also as one continuous unit. The gear encompasses the central portion of the two “/” and “\” beams, forming the central connection of the “X.” A cloth cushion covers the entire structure. Foam padding filler is available.

### Details of the Invention Operation/Functions

The function will be to maintain the position of the human head while resting in a neutral position. No aspect of the front of the neck will be contacted. No nerve or large vessel will be contacted. Specialist input from medical professionals was sought for expert guidance on this design. The device rests upon the shoulders and a gear, provided at the midline crossing arms, serves as a method of raising the upper platforms into position upon the jaw, thereby serving as a support strut to maintain the head in a neutral position when the user sleeps.

The clavicle processes will serve as a base foundation for support of the upper structures, holding the top of the clavicle to facilitate this and holding the front of the clavicle to prevent backward motion of the device. The mandible processes will cradle the angle of the jaw, holding the head in the neutral position as the person sleeps, preventing downward head movement as well as jaw collapse backward and into the airway while the person is asleep. The 10-degree upward angle will further facilitate maintenance of the device's position while in place. The connecting arms and angle of approach avoid at-risk anatomical structures while wearing the device.

The gear will function as a means of fitting the device to the user. The “X” will be applied in a collapsed form, with the mandible processes then raised to the neutral, comfortable position of the user by activating the gear and manually advancing/raising the “X” from its collapsed form to its taller positioning beneath the at-rest jaw, with the users mouth closed at the time of positioning. The patient will be free in neck range of motion to approximately 30-degrees, breathing and speaking while wearing the device.

### Remarks/Discussion

It is not obvious that these prior art applications/patents accomplish the same goal. The prior art appears to stabilize the cervical spine, while JawBridge facilitates mobility and remains clear of vital anterior neck structures. Thank you for the consideration.

### Patent Name: Jaw Bridge

1. A support apparatus for the head, serving an overall function of maintaining the head in a neutral position while at rest, comprising: A foot process consisting of a stabilizing foundation into the clavicle and shoulder; a jaw process consisting of a stabilizing foundation onto the angle of the jaw in a passive jaw thrust position; and shafts connecting said processes.
